(19) United States
(12) Patent Application Publication
(10) Pub. No.: US 2003/0028156 A1
(43) Pub. Date: Feb. 6, 2003

(54) Fluid connector devices and methods of use

(75) Inventor: Rena Juliar, Corona, CA (US)

Correspondence Address: OPPENHEIMER WOLFF & DONNELLY LLP, 840 NEWPORT CENTER DRIVE, SUITE 700, NEWPORT BEACH, CA, 92660, US

(73) Assignee: Nexell Therapeutics, Inc.

(21) Appl. No.: 10/143,714

(22) Filed: May 7, 2002

Related U.S. Application Data
(60) Provisional application No. 60/289,059, filed on May 7, 2001.

(51) Int. Cl. A61M 35/00; F16L 31/00

(52) U.S. Cl. 604/310

(57) ABSTRACT

A device and method of use are disclosed to facilitate fluid transfers during sterile closed system processing procedures. The device is configured to create connections for the transfer of fluid between various components, along a closed sterile fluid passageway and, eventually, either to a fluid container or to a patient. As a result, the device and method of use of the present invention reduce the risk of contamination to the fluids, such as reagents, medicaments and cellular products, and increase user or technician safety during processing procedures.

### CROSS REFERENCE TO RELATED APPLICATIONS

The present application claims priority to U.S. Provisional Patent Application No. 60/289,059, filed May 7, 2001, whose contents are fully incorporated herein by reference.

### BACKGROUND OF THE INVENTION

The medical industry utilizes a wide variety of solution containers, container caps, connectors and tubing sets to create connections for the transfer of fluid between various components, along a fluid passageway and, eventually, either to a fluid container or to a patient. Examples of such connections include, but are not limited to, the processing of blood and its fractions, mixing of sterile solutions and connecting Foley catheters with urinary drainage bags. In order to maintain the sterility of the fluid pathway and minimize the risk to the operator (e.g., needle sticks, biohazard exposure, etc.), these connections should be designed to be closed (i.e., without open exposure of the fluid pathway to air).

In closed system cellular processing, for example, single use sterile disposable sets (e.g., bags, tubing, etc.) are joined together to enable the closed handling of biological fluids. One or more access points into sterile disposable sets are key to user convenience and system flexibility in closed system processing. All additions and removal of fluid (e.g., sampling, etc.) and connections of sets or other components for a next processing step are made via these access points. Access points are typically terminal tubing connectors such as luer fittings, spike couplers and bag ports (e.g., spikable membrane ports).

Luer connectors are widely used in the medical industry for making a connection between medical devices to establish a fluid passageway. In general, a luer connection assembly includes a male luer tip component or fitting having a frustoconical shape that is inserted into a female luer component or fitting having a frustoconically shaped receiving cavity. The opposing conical surfaces of the luer fittings come into contact and form a sealed friction fit assembly.

These connection assemblies and associated components are typically packaged in sterile packaging and include caps or protectors to maintain the sterility of the fluid pathway prior to use. However, at the time the actual connection between components is made, the fluid contacting surfaces and passageways are open to the environment. As a result, these connections must frequently be made inside a laminar flow hood to mitigate the fluid contamination risk.

Another example of an access point or terminal connector is a spike coupler. A tubing set having a spike coupler as its terminal connector may be fluidly connected to a bag having a spikable membrane port. In general, a spike coupler assembly includes a spike component having a needle-like shape with a beveled tip that is inserted into a spikable membrane port generally having a cylindrically shaped receiving cavity or opening. The opposing surfaces of the spike coupler and membrane port come into contact and form a sealed friction fit assembly. The spike coupler is used to access the contents of the bag through the membrane port. When the spike is inserted into the port, the membrane is broken causing the fluid to flow from the container through the spike and into the tubing set. In contrast to the above-described luer connectors, sets joined via spike couplers and spikable membrane ports maintain an aseptic fluid pathway. This processing system has the advantages of reduced potential for contamination of the contained fluid and reduced potential for operator exposure to the biohazards presented by such fluids.

Common problems or drawbacks associated with more complicated processing using sets with spike couplers include exceeding the number of available access points on a particular disposable set or similar component. In these situations, it is often necessary to transfer the fluid to a new set in order to increase accessibility. Moreover, sets or containers having incompatible terminal connectors, such as both sets terminating in spike couplers, further adds to the difficulties encountered during more complicated processing and may render the closed system fluid transfer process an impossibility altogether.

In view of the foregoing, it would be desirable to have a unitary one-piece adapter or connector which enables the connection of two incompatible spike couplers. The adapter should also include an integral cover to maintain sterility when the adapter is not in use. As such, it is also desirable that the lumen and/or passageway through the adapter that forms a portion of the fluid passageway be sterile prior to use and maintain sterility, such as by being sealed against microbial ingress, during connection and disconnection of the spike couplers.

It is a further object of the present invention to provide a connector which is capable of being manufactured at high speeds and low cost. Generally the lower the number of parts making up a component, the lower the number of required molds and high speed assembly devices, both of which generally translate to lower capital expenditures and therefore lower costs. It is a related object of the present invention to provide a connector which may be manufactured with a very low number of potential defects.

Depending on the application, many other features may also be desirable. Dead spaces or voids within the connector which cannot be “flushed” and in which stagnant fluid can collect to form a media for microbial growth should be minimized or eliminated. It is a related object of the present invention to provide a connector which forms a sealed fluid path such that a minimum number of microbes enter the fluid path during operation using aseptic techniques. Also, priming volume for the connector should be minimized.

It is still a further object of the present invention to provide a connector which minimizes or eliminates flow restrictions for the flow of fluid through the connector. It is another object of the present invention to provide a connector device which is capable of providing for a large number of connections and disconnects while maintaining the ability to seal against fluids under pressures typically found in an administration set.

For some processing procedures, it is necessary to transfer the fluid from a manufacturer provided open container, such as a bottle, to a differently configured closed container, such as a bag, to perform the closed system processing procedure. In this instance the user must pipette or decant the bottle contents into the secondary container inside of a laminar flow hood. This is an open and laborious process which, as a consequence, increases the potential for contamination of the fluid and exposure to the operator. In this instance, there is a need for a coupler that will facilitate the transfer in a closed and sterile fashion.

An analogous need also exists in open system centrifugation separation procedures. It is common in the medical, cell processing and other fields to separate cells from suspending fluids via centrifugation. This operation is typically required when washing cells. The fluid component of the separation is aspirated or decanted from the pellet of cells resulting from the applied centrifugal forces. While sample volumes of 500 ml or greater may be washed in bags using closed automated systems currently available on the market, smaller volumes are typically washed using commercially available open conical tubes. This is due to the cell loss associated with bag use. In particular, cells become trapped along the seams of bags and the flexible materials/structure of bags cause their contents to move during handling, thereby dislodging the cells from their concentrated pellet formation. Therefore, a connector or system that enables the integration of rigid open vessels with closed flexible containers is needed.

Although rigid containers overcome these shortcomings typically associated with flexible bags, the use of rigid containers requires access to air. This is due to the fact that it is not possible to add or subtract fluids from such a container and maintain a constant volume inside the rigid closed container. Typically, a volume of air is needed to compensate for removed/added fluids. For example, the removal of 200 ml of fluid results in the addition of an equal amount of air into the container and, thereby, into contact with the fluid path. As a result, the addition of air into the container potentially increases the risk of fluid and system contamination. Therefore, a connector or system that enables the addition and/or release of air/gases into a container without increasing the risk of fluid and system contamination and operator exposure is needed.

### BRIEF SUMMARY OF THE INVENTION

In view of the foregoing, it is an object of the present invention to provide a unitary one-piece adapter or connector which enables the connection of two incompatible spike couplers and requires minimal priming volume.

It is a further object of the present invention to provide an adapter with an integral cover that maintains sterility when the adapter is not in use.

It is a further object of the present invention to provide an adapter having a lumen and/or passageway that forms a portion of the fluid passageway through the adapter, wherein the adapter is sterile prior to use and maintains sterility during connection and disconnection of the spike couplers.

It is a further object of the present invention to provide a connector that is capable of being manufactured at high speeds and low cost.

It is a further object of the present invention to provide a connector which may be manufactured with a very low number of potential defects.

It is a further object of the present invention to provide a connector which forms a sealed fluid path such that a minimum number of microbes enter the fluid path during operation using aseptic techniques.

It is a further object of the present invention to provide a connector which minimizes or eliminates flow restrictions for the flow of fluid through the connector.

It is a further object of the present invention to provide a connector which is capable of providing for a large number of connections and disconnects while maintaining the ability to seal against fluids under pressures typically found in an administration set.

It is a further object of the present invention to provide a connector that will facilitate the transfer of fluids in a closed sterile fashion.

It is a further object of the present invention to provide a connector or system that enables the integration of rigid open vessels with closed flexible containers.

These and other objects not specifically enumerated her are addressed by the present invention which in at least one embodiment may include a fluid connector comprising a first conduit, a second conduit, and a cylindrically shaped sleeve positioned in fluid communication with said first and second conduits. The connector may further include a membrane located within each of the conduits and forming a closed chamber extending between the membrane of the first conduit and the membrane of the second conduit. In general, the connector forms a closed fluid pathway and provides a sufficient fluid flow path for connecting devices.

The present invention may further include a method of using a fluid connector comprising inserting a spike coupler of a first tubing set into a first conduit of a connector and advancing the spike coupler within the first conduit until at least a tip of said spike coupler penetrates a membrane of the first conduit. The method further comprises inserting a spike coupler of a second tubing set into a second conduit of the connector and advancing the spike coupler within the second conduit until at least a tip of said spike coupler penetrates a membrane of the second conduit, thereby forming a closed, aseptic fluid pathway through the connector and tubing sets.

### BRIEF DESCRIPTION OF THE DRAWINGS

Other features and advantages of the present invention will be seen as the following description of particular embodiments progresses in conjunction with the drawings, in which:

FIG. 1 is a perspective view of a dual port membrane adapter device in accordance with an embodiment of the present invention;

FIG. 2 is illustrates the assembly and internal components of the adapter device in accordance with an embodiment of the present invention;

FIG. 3 is an end view of the adapter device in accordance with an embodiment of the present invention;

FIG. 4 is an end view of an alternate embodiment of the device of the present invention;

FIG. 5 is a sectional view of the adapter device in accordance with an embodiment of the present invention;

FIG. 6 illustrates a cap-shaped device in accordance with an embodiment of the present invention;

FIG. 7 is a perspective view of the cap-shaped device in accordance with an embodiment of the present invention;

FIG. 8 is an alternate view of the cap-shaped device in accordance with an embodiment of the present invention;

FIG. 9 illustrates an alternate embodiment of the cap-shaped device in accordance with the present invention;

FIG. 10 illustrates a method of using the adapter in accordance with an embodiment of the present invention; and

FIG. 11 illustrates a method of using the cap-shaped device in accordance with an embodiment of the present invention.

### DETAILED DESCRIPTION OF THE INVENTION

The following description and figures are meant to be illustrative only and not limiting. Other embodiments of this invention will be apparent to those of ordinary skill in the art in view of this description.

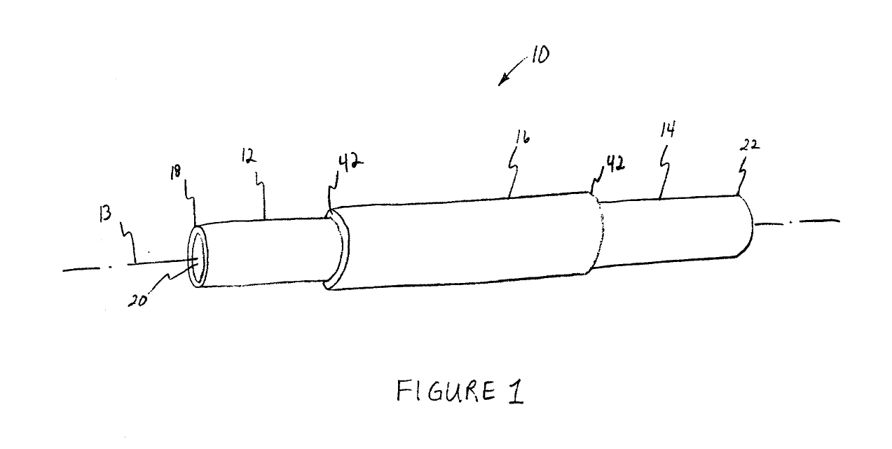

Referring to FIG. 1, an embodiment of a fluid transfer device in accordance with the present invention includes a connector or adapter 10 having a first cylindrically shaped conduit 12, a second cylindrically shaped conduit 14, and a hollow, cylindrically shaped sleeve 16 located between the first and second conduits 12,14 of the adapter 10. Each conduit 12,14 and the sleeve 16 include a first end, a second end and a passageway extending therebetween for the passage of fluid. The conduits 12,14 and sleeve 16 are aligned along the same longitudinal axis 13 and are configured in fluid flow relationship with each other.

In one embodiment of the device, the first end 18 of the first conduit 12 includes a fluid inlet port 20 and the second end 22 of the second conduit 14 includes a fluid outlet port 24. Alternatively, the first end 18 of the first conduit 12 may include a fluid outlet port 20 and the second end 22 of the second conduit 14 may include a fluid inlet port 24. In general, the conduits 12,14 are configured for quick connection for releasably engaging a terminal end, such as a spike coupler, of a tubing set.

As shown in FIGS. 1, 2 and 3, each conduit 12,14 is approximately 1.00±0.05 inch (25.40±1.27 mm) in length LC. The tubular wall 26 forming the structure or framework of each conduit 12,14 may include a smooth internal surface 28, a smooth external surface 30 and a wall thickness TC. In one embodiment of the adapter 10, the wall thickness TC, internal diameter IDC and external diameter EDC of each conduit are approximately within the range of 0.030±0.005 inch (0.762±0.127 mm), 0.190±0.005 inch (4.826±0.127 mm) and 0.250±0.005 inch (6.350±0.127 mm), respectively. The diameters and wall thickness may either be uniform or variable along the longitudinal length of each conduit 12,14. A particular choice of wall thickness, diameters and length depends on the configuration of the mating terminal end intended for connection to the adapter 10. In other words, the configuration of each conduit 12,14 can vary according to the intended procedure, mating connection and usage.

Similarly, the dimensional configuration of the sleeve 16 of the adapter 10 should be appropriately sized to surround and overlap portions of the conduits 12,14 and to provide a sufficient flow path for connecting devices (e.g., tubing set spike couplers). In one embodiment, the length LS of the device sleeve 16 may be approximately within the range of 1.50±0.05 inch (38.10±1.27 mm). In addition, the overlap portions 32 of the conduits 12,14 and sleeve 16 may each be within the range of 0.25±0.05 inch (6.35±1.27 mm).

The tubular wall 34 forming the structure of the sleeve 16 may include a smooth internal surface 36, a smooth external surface 38 and a wall thickness TS. In one embodiment, the wall thickness TS, internal diameter IDS and external diameter EDS of the sleeve 16 are approximately within the range of 0.030±0.005 inch (0.762±0.127 mm), 0.245±0.005 inch (6.223±0.127 mm) and 0.305±0.005 inch (7.747±0.127 mm), respectively. As with the conduits 12,14, the diameters and wall thickness of the sleeve 16 may either be uniform or variable along the length of the sleeve. In general, the internal diameter IDS of at least a portion of the sleeve 16 should be appropriately sized to match at least a portion of the external diameter EDC of each conduit 12,14 to ensure that there is a snug and conforming fit and contact between the opposing external and internal surfaces 30,36 of each conduit 12,14 and sleeve 16, respectively. Further, the internal diameters of both the conduits 12,14 and sleeve 16 should be large enough to adequately support an unrestricted flow of fluid therethrough. Overall, the design of the adapter 10 should facilitate the connection of tubing lines that terminate in spike couplers.

In an alternate embodiment of the invention (not shown), the opposing external and internal surfaces 30,36 of each conduit 12,14 and sleeve 16, respectively, may be textured to increase the surface area and, thereby, further enhance the contact characteristics of the surfaces of the friction fit assembly 10. In yet another embodiment of the device 10, the opposing external and internal surfaces 30,36 of each conduit 12,14 and sleeve 16, respectively, may be threaded for threaded engagement between the components of the adapter 10. Alternative engagement features and configurations including, but not limited to, ridges, channels, grooves, bumps, indents, prongs, rods, tabs, flanges, chamfers, and threads are also included within the scope of the claimed invention.

The conduits 12,14 and sleeve 16 of the adapter 10 may be further secured together via ultrasonic welding. Additional techniques and methods used to secure together the components of the assembly 10 include, but are not limited to, frictional welding, chemical bonding, heat shrinking, and fusing. These and other techniques and methods not specifically disclosed herein but known to those skilled in the art are also included within the scope of the claimed invention.

In addition to the particular dimensional attributes of the conduits 12,14 and sleeve 16, the material characteristics of the adapter 10 are also important to achieve the desired performance features of the device 10. A variety of materials may be used to fabricate the adapter 10 of the present invention. These materials include, but are not limited to, plastics (e.g., polyethylene, polyvinyl chloride, polycarbonate, etc.), silicone, stainless steel, metals, and ceramics, including combinations thereof. In general, the adapter materials should be sterilizable, biocompatible and non-pyrogenic.

Although the adapter 10 may be constructed as a rigid, flexible or semi-flexible connector, it is preferred that the materials together with the structural design of the adapter 10 provide sufficient strength and structural integrity to avoid kinking, collapse or restriction of the closed fluid flow path of the conduits 12,14 and sleeve 16. In one embodiment of the invention, the adapter 10 may be configured as single use, disposable connector. Alternatively, the adapter 10 may be configured as a multiple use and re-sterilizable device.

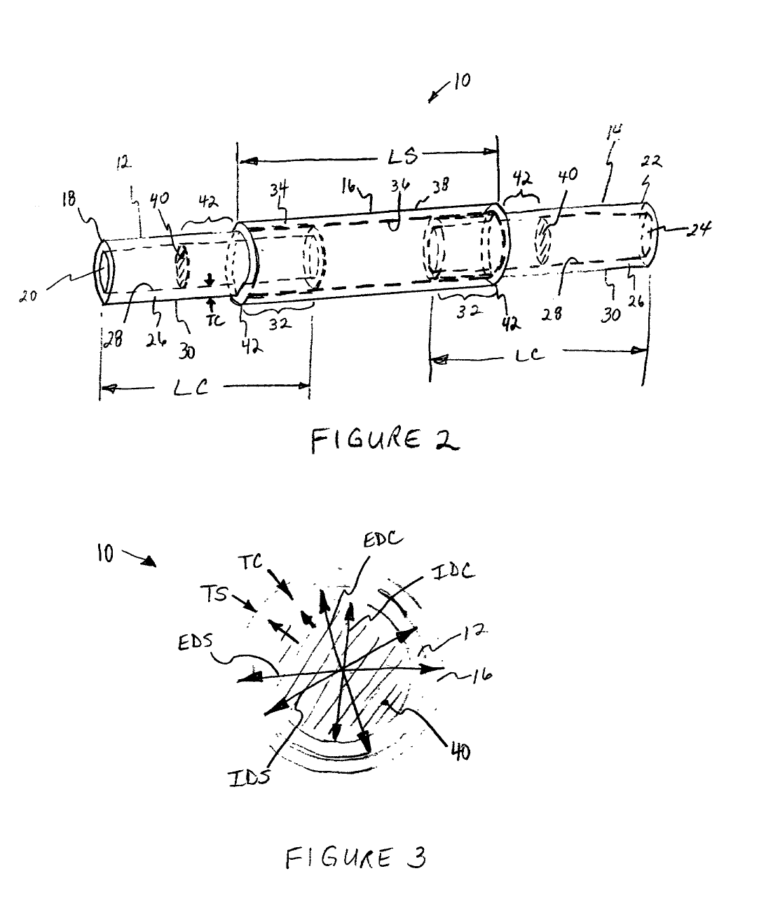

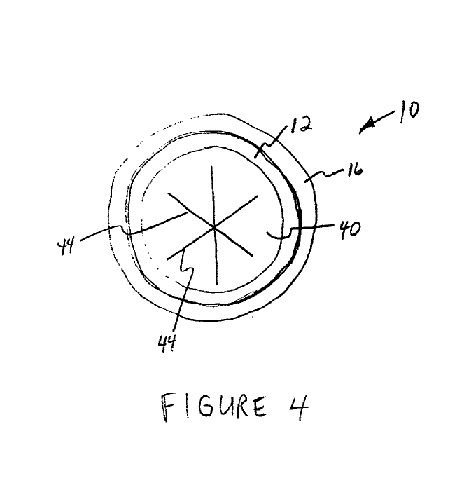

Referring to FIGS. 2 and 3, each conduit 12,14 of the adapter 10 includes a transverse diaphragm or membrane 40 adapted for breakage by a spike coupler from a tubing set. The membrane 40 may be located anywhere along the longitudinal length of the conduits 12,14 and, preferably, is located near the mid-section of each conduit 12,14. In one embodiment of the invention, the membrane 40 is located approximately within the range of ±0.125 inch (±3.1750 mm) from an end 42 of the sleeve 16 when the conduit 12,14 is housed within the sleeve 16. As a result, the two membranes 40 of the adapter 10 form a closed chamber that extends along the length LS of the sleeve 16 and a portion 42 of the length LC of each conduit 12,14.

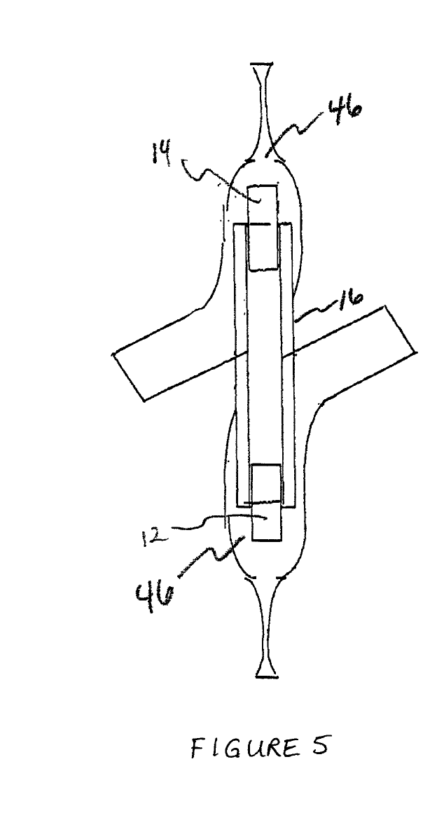

In one embodiment of the invention, the membrane 40 is formed as a solid, resilient barrier capable of being pierced or broken when a terminal connector (e.g., spike coupler) is properly seated within the adapter 10. In an alternate embodiment, shown in FIG. 4, the membrane 40 may include one or more slits 44 forming a valve-like element capable of being re-sealed in its closed configuration. For example, when a spike coupler penetrates the membrane 40, the slit(s) 44 spread apart and form an opening in the membrane 40 that substantially surrounds a portion of the spike coupler. The resulting connection between the spike coupler of the tubing set and the conduit 12,14 of the adapter 10 forms a closed fluid pathway, thereby minimizing, if not altogether eliminating, contamination risks. When the spike coupler is removed or disconnected from the adapter 10, the slit(s) 44 return to their original closed configuration and, essentially, re-seal the opening in the membrane 40. As such, the adapter 10, particularly the chamber of the adapter 10, maintains a continuous, closed, contamination-free system that seals the fluid passageway from the environment before, during and after a connection is formed.

Each membrane 40 of the present invention may be fabricated from essentially the same materials of the conduits 12,14 and sleeve 16, as previously described. In an alternate embodiment, the membranes 40, conduits 12,14 and/or sleeve 16 of the device 10 may be fabricated from dissimilar materials. In a preferred embodiment, the membrane material is resilient and includes memory-characteristics that enable the membrane to return to its original configuration after use and adequately seal the fluid path of the system.

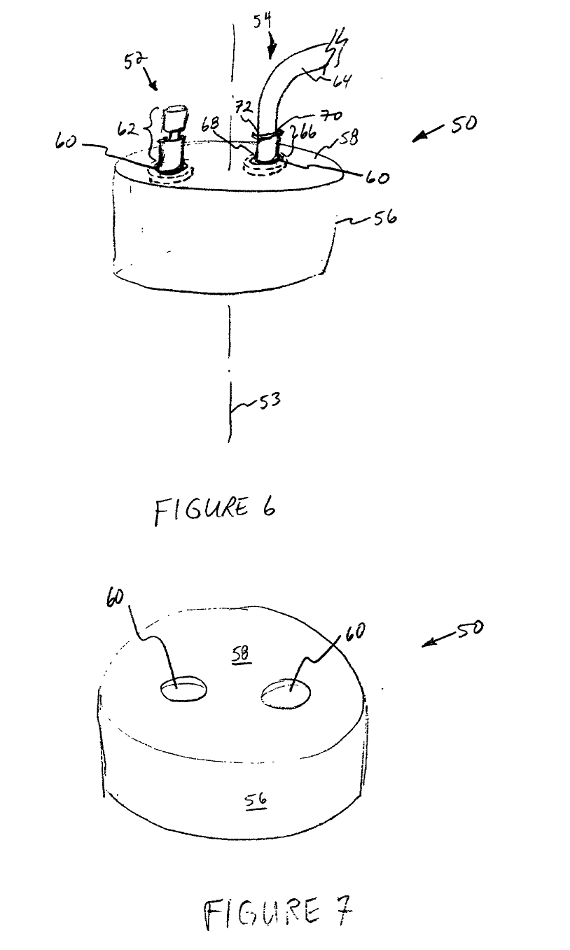

As shown in FIG. 5, the adapter 10 of the present invention may include one or more covers or tip protectors 46 to shield the fluid inlet port and fluid outlet port of the conduits 12,14 from damage and contamination risks. These protectors maintain the sterility of the membrane portion of the connector during storage and up to the time of use.

As noted in the Background of the Invention as set forth above, in addition to connectors, other types of fluid transfer devices used in the medical industry include container caps and closed container devices. Referring to FIG. 6, a cap-shaped device in accordance with the present invention may include a sterile filtration vent 52 and a tubing segment assembly 54. In general, the cap-shaped device 50 includes a cylindrical neck 56 and a substantially planar surface 58 positioned perpendicular to the longitudinal axis 53 of the neck 56 and device 50.

To accommodate a variety of processing procedures and container configurations, the cap-shaped device 50 may be configured as a rigid or semi-flexible component. In one embodiment, the cylindrical neck 56 of the device 50 is configured for attachment to a bottle, conical tube, or other fixed volume fluid container. However, it should be noted that alternate configurations of the neck 56 including, but not limited to, square-shaped, oval, and polygonal are known in the art and, as such, are also included within the scope of the claimed invention.

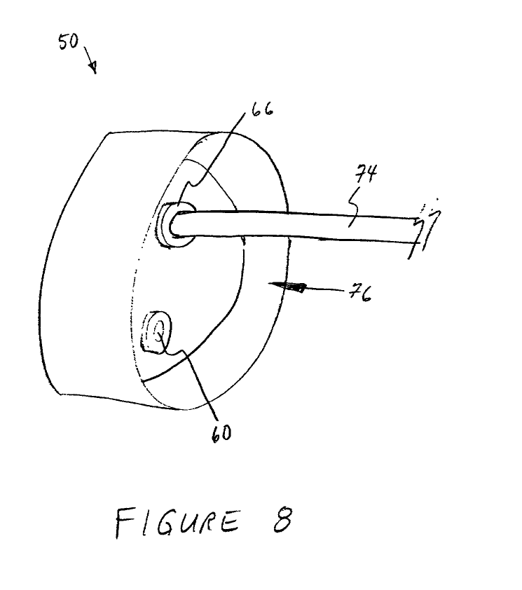

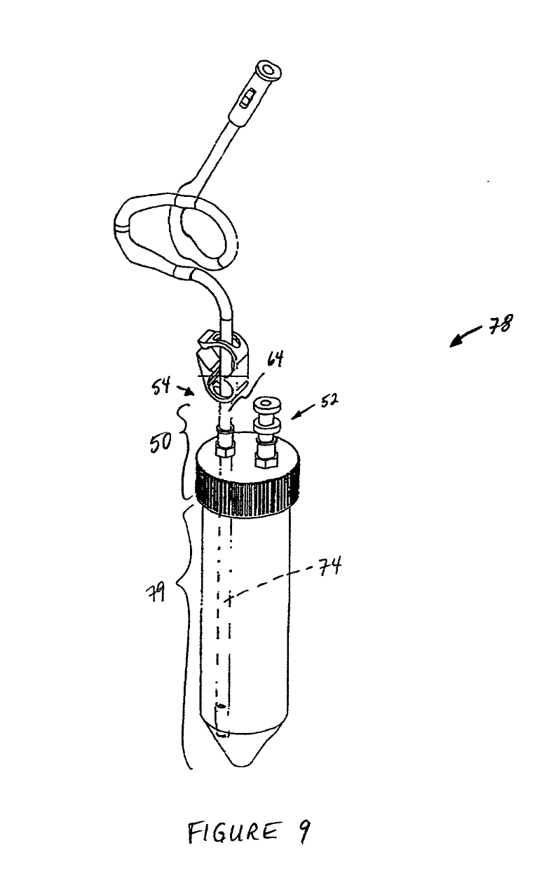

Two or more access ports or openings 60 are provided on the surface 58 of the device 50, as shown in FIG. 7. In one embodiment of the invention, a sterile filtration vent 52 may extend from the first access port 60 and a tubing segment assembly 54 may extend from the second access port 60. Referring to FIG. 6, the sterile filtration vent 52 may include a sterile, tubular or luer-shaped element 62 having a filter (not shown) located within the interior cavity of the element 62. Before ambient air may enter or leave the system, it must first pass through the filer of the vent 52. As such, the sterile filtration vent 52 accomplishes several goals of the present invention. First, by filtering ambient air before it enters the system, the sterile filtration vent 52 maintains system sterility and reduces the probability of fluid and system contamination. In addition, since entrapped gases/air must also pass through the filter before leaving the system, user exposure to such potentially toxic and harmful gases/air is significantly reduced or completely eliminated by virtue of the vent 52. Further, with respect to fluid transfer operations using rigid containers, the sterile filtration vent 52 allows air and gases to enter/escape through the vent 52 and into/out of the closed rigid container to compensate for removed/added fluids, while maintaining sterility of the system via the filter.

Referring to FIGS. 6 and 8, the tubing segment assembly 54 of the device 50 of the present invention may include a segment of tubing 64 and a sterile tubular or luer-shaped element 66. A first end 68 of the luer-shaped element 66 may extend from the second access port 60 of the device 50 and a second end 70 of the luer-shaped element 66 may be in communication with a first end 72 of the tubing segment 64. Extending from the second end (not shown) of the tubing segment 64 may be a terminal end of another closed system sterile disposable set used to access to the contents of the container.

In one embodiment of the invention, a second tubing segment 74 may also extend from the second access port 60 and/or first end 68 of the luer-shaped element 66 within the interior portion 76 of the cap-shaped device 50, as shown in FIG. 8. This second tubing segment 74 may be used as a straw or wick to access the contents of the container during use of the device 50, as further described below. Depending on the particular function or procedure, the length of the second tubing segment may vary. For example, in a large volume bottle cap, the tube 74 may extend to the bottom of the bottle and be configured with a beveled or other similar non-blunt end. This design would facilitate removal of the contents by pumping when the container is in an upright position.

Analogously, when the cap-shaped device 50 is fitted onto a centrifuge tube with a conical bottom, the second tubing segment 74 may extend to varying levels or depths towards the conical bottom. In essence, the level or lengths of the second tubing segment 74 dictates the lowest level of fluid which may be aspirated from a container.

In an alternate embodiment, the second tubing segment 74 may extend through the second access port 60 of the device 50 (not shown). For this embodiment, the second tubing segment 74 may be mated to the cap 50 in such a way as to enable a hermetic seal while allowing for user manipulation of tubing depth within the container to thereby access a variety of fluid levels.

As with the adapter 10, the connector or cap-shaped device 50 of the present invention may be fabricated from a variety of materials, such as those previously described herein. In general, the device materials should be sterilizable, biocompatible and non-pyrogenic. Together with the materials, the particular dimensional characteristics of the device 50 should be optimized to achieve the desired performance features of the device 50, such as desired fluid flow rates and sufficient venting. In particular, the vent 52 may be configured to include an adequate flow-through and filtration area to enable free fluid flow from the tube 54. In addition, the tubing segments 64,74 may be configured as flexible, semi-flexible or rigid components, depending upon the desired performance characteristics of the device 50. Generally, in the field of closed system cellular processing, the device 50 should be configured to allow fluid transfers and processing to be conducted in the open laboratory environment, without the risk of contaminating the reagents and cellular products and exposing technicians to contamination during the procedure.

In one embodiment of the invention, the cap-shaped device 50 may be supplied to users in several conventional container (e.g., bottle, bag, tube, etc.) bottle sizes to accommodate a variety of bottled reagents. In another embodiment, the cap 50 may be pre-fabricated onto a container and provided as an integral device. In this regard, FIG. 9 depicts an example of what this embodiment of a capped-container device 78 may look like in accordance with the present invention. In essence, the device 78 includes the cap-shaped device 50 and a container 79.

Although the container 79 depicted in FIG. 9 is a conical tube, any of a variety of containers including, but not limited to, bottles and bags may be used and are also included within the scope of the claimed invention. In one embodiment, this container 79 may be supplied in an unfilled or empty state to enable user addition/subtraction of fluid. In addition, the cap-shaped device 50 may be either permanently or removably attached to the container 79. In an alternate embodiment, the container 79 may be supplied pre-filled with a solution, reagent or other desired fluid to bypass the need for cap placement entirely. Similar to the previous embodiment, the pre-filled cap-shaped device 50 may be either permanently or removably attached to the container 79.

Method of Use

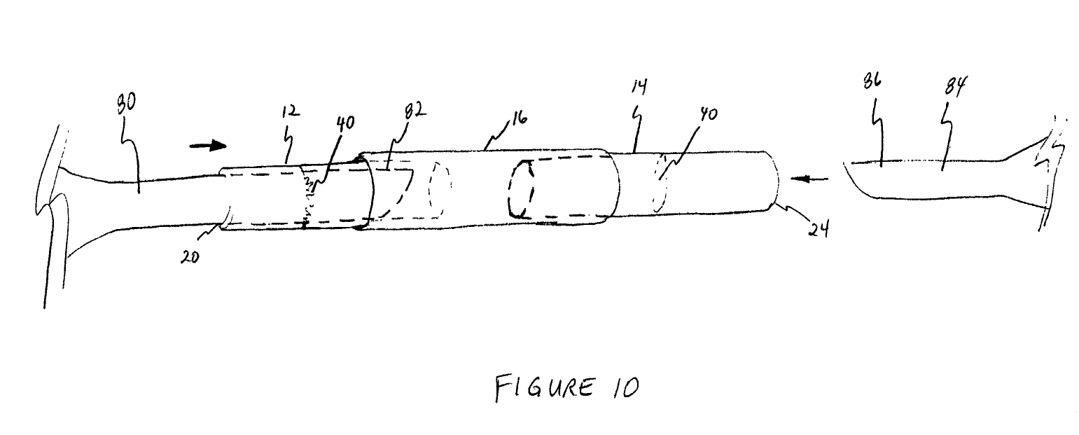

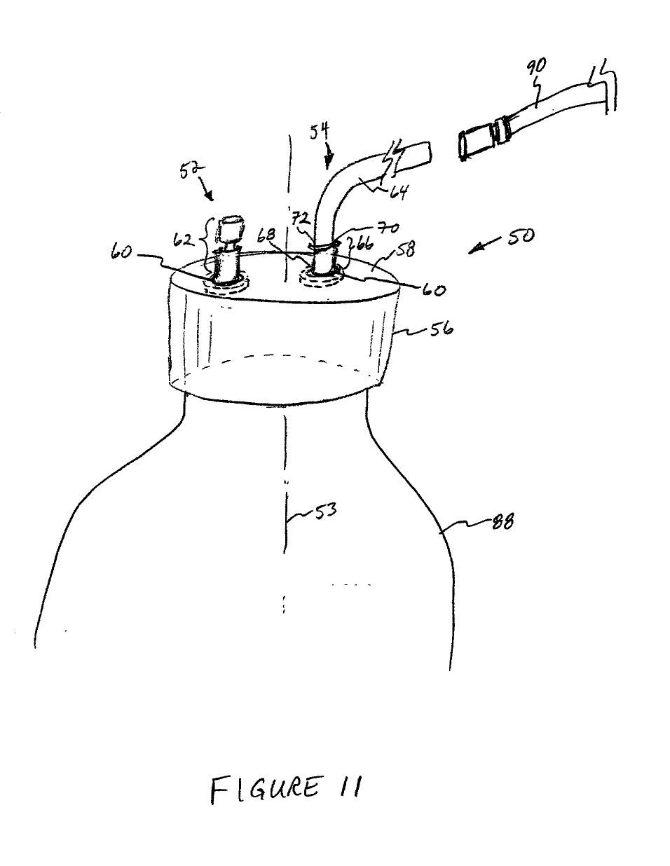

In one embodiment of the invention, the double membrane port adapter 10 may be used in closed system cellular processing. As shown in FIG. 10, one step of a typical cellular processing procedure may require two single use sterile disposable sets (e.g., tubing sets terminating in spike couplers) to be joined together for a fluid transfer operation. To join the sets, a spike coupler 80 of a first tubing set is inserted along the longitudinal axis of the adapter 10, through a port 20 and into a first conduit 12 of the device 10. The spike coupler 80 is then advanced further within the conduit 12 until at least the tip 82 of the spike coupler penetrates through the membrane 40 of the conduit 12 and the spike coupler 80 is properly seated within the adapter 10. At this point in the procedure, the fluid pathway of the first tubing set remains in a closed, sealed configuration due to the unbroken membrane 40 of the second conduit 14 of the adapter 10.

Next, a spike coupler 84 of a second tubing set is inserted along the longitudinal axis of the adapter, through a port 24 and into a second conduit 14 of the device 10. The spike coupler 84 is then advanced further within the conduit 14 until at least the tip 86 of the spike coupler 84 penetrates through the membrane 40 of the conduit 14 and the spike coupler 84 is properly seated within the adapter 10 (not shown). Penetration of the second spike coupler 84 through the membrane 40 of the second conduit 14 creates a closed, continuous, aseptic fluid pathway and, thereby, enables fluid to be transferred via the sets.

Closed system cellular processing methods may also require the transfer of fluid into or out of containers. In this instance, a cap-shaped device 50 may be used to provide added convenience for the user and processing flexibility. During use, a user of the device 50 would replace the manufacturer provided cap on a container with the cap-shaped device 50 of the present invention while under a laminar flow hood. As shown in FIG. 11, the capped container 88 could then be removed to the general laboratory environment, where all fluid transfers of the processing procedure may be performed while using the device 50. Next, the tubing segment 64 on the cap-shaped device 50 could be connected to other closed system sterile disposable sets 90. As a result, fluid could then be transferred from the bottle 88 to the closed system sterile disposable set 90 through the tubing segment 64 via gravity by inversion of the bottle 88. In addition, if a rigid container is used, a volume of ambient air will enter the container/bottle 88 via the sterile filtration vent 52 to replace the lost volume of fluid during the fluid transfer operation. As such, the tubing segment assembly 54 forms a closed system fluid pathway, together with the filtration vent 52, for maintaining the sterility of the system.

In an alternate embodiment, the fluid may be pumped out of the bottle 88, through the tubing segment 64 and into the disposable set 90. In yet another embodiment of the invention, the cap-shaped device 50 may be configured with a second tubing segment 74 inside the bottle. As a result, the bottle 88 could remain in an upright position and the fluid could be pumped out of the bottle 88 via the second tubing segment 74, through the tubing segment 64 and into the disposable set 90.

As a further convenience for the user and means to maintain system sterility, the processing procedure may be carried out using the cap-shaped device 50 configured onto a conical shaped tube/container 79, as shown in FIG. 9. During the processing procedure, a cell suspension may be added to the container 79 by connecting the tubing segment 64 to another closed sterile set. The device 78 would be sealed (reversibly or irreversibly) and the whole apparatus 78 placed into a centrifuge. Next, the apparatus 78 would be centrifuged resulting in pelletization of the heavier cells or other particles from the fluid. After centrifugation, the apparatus 78 would be removed from the centrifuge and another connection would be made to the tubing segment 64, typically via an alternate distal connector. The fluid portion of the suspension may then be aspirated via the second tubing segment 74 into a closed receiving container (not shown) via gravity and capillary action. This process of aspirating the fluid from the container 79 may be accomplished by placing the receiving container a sufficient distance below the conical shaped tube 79 to draw off the fluid. Residual fluid in the bottom of the container 79 may be aspirated by slightly tipping the container 79 and without disturbing the cell pellet.

As previously described, the length of the second tubing segment 74 may be configured as desired to remain near the level of a cell pellet, yet not allow cells to become lodged there during centrifugation. It is clear that the tubing length might be longer to accommodate tubes of greater length. In additional embodiments of the invention, caps 50 (or full assemblies 78) may be made with different tubing lengths. As such, the user would simply decide which embodiment to use based on the chosen application.

In view of the foregoing, the cap-shaped device 50,78 of the present invention facilitates the addition of the bottled reagent or fluid to closed sets and, thereby, reduces overall costs of processing procedures. In addition, the device 50,78 enables processing in an open laboratory environment by providing a closed system fluid path that reduces the risk of contamination to the reagents and cellular products and increases user or technician safety during processing procedures. In particular, the device 50,78 reduces the potential of exposure of the operator to potentially biohazardous materials.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A fluid connector comprising:
a first conduit;
a second conduit;
a cylindrically shaped sleeve positioned in fluid communication with said first and second conduits; and
a membrane located within each of said conduits and forming a closed chamber extending between said membrane of said first conduit and said membrane of said second conduit.

2. The fluid connector of claim 1 wherein said conduits are configured for quick connection for releasably engaging a terminal end of a tubing set.

3. The fluid connector of claim 2 wherein said connection forms a closed fluid pathway.

4. The fluid connector of claim 2 wherein said terminal end is a spike coupler.

5. The fluid connector of claim 1 wherein said conduits and sleeve provide a sufficient fluid flow path for connecting devices.

6. The fluid connector of claim 5 wherein said connecting device terminates in a spike coupler.

7. The fluid connector of claim 1 wherein said conduits and sleeve include smooth internal surfaces.

8. The fluid connector of claim 1 wherein said conduits and sleeve are fabricated from materials that are sterilizable, biocompatible and non-pyrogenic.

9. The fluid connector of claim 1 wherein said membrane is adapted for breakage by a spike coupler from a tubing set.

10. The fluid connector of claim 1 wherein said membrane is formed as a solid, resilient barrier capable of being pierced or broken.

11. The fluid connector of claim 10 wherein said membrane is pierced or broken when a terminal connector is properly seated within the fluid connector.

12. The fluid connector of claim 11 wherein said terminal connector is a spike coupler.

13. The fluid connector of claim 1 wherein said membrane includes one or more slits.

14. The fluid connector of claim 13 wherein said slits form a valve-like element capable of being resealed in a closed configuration.

15. The fluid connector of claim 13 wherein a spike coupler penetrates said membrane via said slits.

16. The fluid connector of claim 15 wherein said spike coupler and fluid connector form a closed fluid pathway.

17. The fluid connector of claim 1 wherein said conduits and sleeve form a continuous, closed, contamination free system that seals a fluid passageway from an environment before, during and after a connection is formed.

18. The fluid connector of claim 1 further comprising one or more covers to protect said conduits from damage and contamination.

19. A method of using a fluid connector comprising:
inserting a spike coupler of a first tubing set into a first conduit of a connector;
advancing said spike coupler within said first conduit until at least a tip of said spike coupler penetrates a membrane of said first conduit;
inserting a spike coupler of a second tubing set into a second conduit of said connector; and
advancing said spike coupler within said second conduit until at least a tip of said spike coupler penetrates a membrane of said second conduit, thereby forming a closed, aseptic fluid pathway through said connector and tubing sets.

20. The method of claim 19 further comprising:
transferring a fluid from said first tubing set to said second tubing set via said connector.
